(19) United States
(12) Patent Application Publication
(10) Pub. No.: US 2021/0365795 A1
(43) Pub. Date: Nov. 25, 2021

(54) METHOD AND APPARATUS FOR DERIVING NEW DRUG CANDIDATE SUBSTANCE

(71) Applicant: MEDIRITA, Seoul (KR)

(72) Inventors: Young Woo PAE, Seoul (KR); Seung-Hyun JIN, Seoul (KR)

(73) Assignee: MEDIRITA, Seoul (KR)

(21) Appl. No.: 17/053,347

(22) PCT Filed: Oct. 21, 2019
(86) PCT No.: PCT/KR2019/013842

§ 371 Date: Nov. 5, 2020

(30) Foreign Application Priority Data
Oct. 11, 2019 (KR) 10-2019-0126489

(51) Int. Cl. G06N 3/08; G06N 3/04; G16C 20/70; G16C 20/50; G16C 20/30

(52) U.S. Cl. CPC G06N 3/088; G06N 3/04; G16C 20/30; G16C 20/50; G16C 20/70

(57) ABSTRACT

A method for deriving a new drug candidate substance that is executed by a computing apparatus is disclosed. The method includes generating a refined knowledge network in which nodes representing biological entities are connected to each other by using a connecting line representing a correlation between the nodes, determining a basic drug for deriving a new drug candidate substance by analyzing drug-disease node pairs existing in the refined knowledge network, and obtaining an analogous substance having a chemical structure analogous to a structure of the basic drug by using an artificial neural network-based structure prediction model. The biological entity includes at least one of a gene, a protein, a metabolite, a symptom, a disease, a compound, and a drug, and a simplified molecular-input line-entry system (SMILES) based character string of the basic drug is input in the structure prediction model.

### TECHNICAL FIELD

The present invention relates to a method and apparatus for developing a new drug, and more particularly, to a method and apparatus for deriving a candidate substance for drug repositioning and predicting physical properties of the candidate substance.

### BACKGROUND ART

It is known that it takes a total of 15 years and costs 2 to 3 trillion won on average to develop a new drug. Among them, it is known that it takes about six years to discover new drug candidate substances before a preclinical trial.

In general, in order to discover new drug candidate substances, which is the first step in the pipeline to develop a new drug, a large number of academically-trained research personnel are going through a process of searching for enormous amounts of information one by one and inferring associations between key biological entities therefrom.

Meanwhile, according to the Life Intelligence Consortium (2017) recently launched in Japan, it is predicted that the time and the cost required to develop a new drug may be shortened to about 40% and be reduced to about 50%, respectively, when artificial intelligence technology is used for the new drug development.

### DISCLOSURE OF THE INVENTION

### Technical Problem

There may be provided a method and apparatus for predicting a chemical structure and a physical property of a candidate substance for drug repositioning based on network analysis of multi-omics data and artificial intelligence technology.

The technical task obtainable from the present embodiment is not limited to the above-mentioned technical task, and other technical tasks may be clearly understood from the following embodiments.

### Advantageous Effects

It is possible to accurately select a candidate drug for drug repositioning based on interaction paths on the multi-omics reflecting complexity of the human body. In addition, by predicting a chemical structure and a physical property of the candidate drug for drug repositioning, it is possible to derive a new drug candidate substance analogous to the candidate drug for drug repositioning and to increase the possibility of success in new drug development.

### BRIEF DESCRIPTION OF THE DRAWINGS

FIG. 1 is a block diagram of a data processing apparatus for discovering a new drug candidate substance, according to an embodiment.

FIG. 2 is a flowchart of a data processing method for discovering a new drug candidate substance by the data processing apparatus, according to an embodiment.

FIG. 3 is a diagram illustrating a predetermined search word input according to an embodiment.

FIG. 4 is a part of a matrix illustrating biological entities extracted in steps S110 and S120 and a correlation between the biological entities, according to an embodiment.

FIG. 5 is a part of an example of a category of the correlation for extracting the matrix of FIG. 4, according to an embodiment.

FIG. 6 is a diagram illustrating a first knowledge network according to an embodiment.

FIG. 7 is a diagram illustrating classification of types of hubs according to participation coefficients (PCs), according to an embodiment.

FIG. 8 is a diagram illustrating a second knowledge network generated from a search word “epilepsy syndrome”, according to an embodiment.

FIG. 9 is a block diagram of a computing apparatus for deriving a new drug candidate substance, according to an embodiment.

FIG. 10 is a flowchart of a method for deriving a new drug candidate substance by the computing apparatus, according to an embodiment.

FIG. 11 is a flowchart of a method for determining a basic drug for deriving a new drug candidate substance by the computing apparatus, according to an embodiment.

FIG. 12 is a diagram illustrating standard scores of proximities of drug-disease node pairs extracted from a refined knowledge network, according to an embodiment.

FIG. 13 is a diagram illustrating a drug-disease node pair with proximity smaller than a reference value and intermediate nodes existing on a path thereto, according to an embodiment.

FIG. 14 is a diagram illustrating a structure prediction model for outputting a substance having a chemical structure analogous to that of a basic drug, according to an embodiment.

FIG. 15 is a diagram illustrating that a basic drug input to a prediction model is transformed into a vector of a reference size, according to an embodiment.

FIG. 16 is a diagram illustrating physical properties predicted for a new drug candidate substance by using physical property prediction models, according to an embodiment.

### BEST MODE FOR CARRYING OUT THE INVENTION

A method for deriving a new drug candidate substance that is executed by a computing apparatus may include generating a refined knowledge network in which nodes representing biological entities are connected to each other by using a connecting line representing a correlation between the nodes, determining a basic drug for deriving a new drug candidate substance by analyzing drug-disease node pairs existing in the refined knowledge network, and obtaining an analogous substance having a chemical structure analogous to a structure of the basic drug by using an artificial neural network-based structure prediction model, wherein the biological entity includes at least one of a gene, a protein, a metabolite, a symptom, a disease, a compound, and a drug, a category of the correlation includes at least one of interact, participate, covariate, regulate, associate, bind, upregulate, cause, resemble, treat, downregulates, palliate, present, localize, include, and express, and a simplified molecular-input line-entry system (SMILES)-based character string of the basic drug is input in the structure prediction model.

The generating of the refined knowledge network may include receiving a search word, extracting at least one biological entity related to the search word from a database (DB) for each biological entity type, extracting a correlation between the search word and the biological entities from a DB for a correlation between biological entities, generating a first knowledge network in which the search word and the biological entities are each set as a node and a plurality of nodes are connected to each other by using a connecting line according to the correlation between the search word and the biological entities or the correlation between the biological entities, calculating a graph theory index of the first knowledge network, and generating a second knowledge network as the refined knowledge network by using a portion of the plurality of nodes that are extracted by using the graph theory index. The search word may include at least one of a gene name, a protein name, a metabolic name, a symptom name, a disease name, a compound name, and a drug name, an identification number may be assigned and a weight is set for each category of the correlation, and the graph theory index is calculated by reflecting the weight set for each category of the correlation, the graph theory index may include at least one of a shortest inter-node path, a clustering coefficient per node, a centrality coefficient per node, and a nature of a hub by node for the plurality of nodes constituting the first knowledge network, and the generating of the second knowledge network may include calculating a standard score per node by using at least one of the shortest inter-node path, the clustering coefficient per node, and the centrality coefficient per node for the plurality of nodes constituting the first knowledge network among the plurality of nodes, deleting a node of which the standard score is less than a threshold value, and deleting the connection associated with the deleted node.

The standard score may be a value obtained by dividing a difference between an index value of a predetermined graph theory index for each of the nodes constituting the first knowledge network and an average index value of a predetermined graph theory index for the plurality of nodes constituting the first knowledge network by a standard error, and the threshold value may be 95% of significance.

The determining of the basic drug for deriving the new drug candidate substance may include calculating standard scores of proximities of the drug-disease node pairs existing on the refined knowledge network, selecting at least one drug-disease node pair with the standard score of the proximity less than a reference value, and determining the drug of the selected at least one drug-disease node pair as the basic drug when a node indicating a disease exists on a path for the drug-disease node pair.

The standard score of the proximity may be a value obtained by dividing a difference between the shortest path of a specific pair of the drug-disease node pairs constituting the refined knowledge network and an average of shortest paths for the nodes constituting the refined knowledge network by a standard deviation.

The obtaining of the analogous substance having the chemical structure analogous to the structure of the basic drug may include converting each of the characters constituting the SMILES-based character string for the basic drug into a vector of a reference size by replacing the character with an index corresponding to the character, and determining an output obtained by inputting the vector into the structure prediction model as the analogous substance.

The determining of the output obtained by inputting the vector into the structure prediction model as the analogous substance may include extracting a feature of the vector by encoding the vector, and outputting a reconstruction vector by decoding the feature.

The artificial neural network may include an input layer, a hidden layer, and an output layer, the number of neurons in the input layer and the output layer may be the same, and the number of neurons in the hidden layer may be less than the number of neurons in the input layer.

Learning about the structure prediction model may be performed based on self-supervised learning in which a synapse of the artificial neural network is updated to generate the same output as the input to the structure prediction model.

The method for deriving a new drug candidate substance may further include predicting a physical property of the analogous substance through an artificial neural network-based physical property prediction model, wherein the physical property includes at least one of solubility, hydration energy, melting point, boiling point, toxicity, electrical stability, excited state property, protein-ligand binding, dissociation constant, and membrane permeability.

The physical property prediction model may be independently generated for each of the physical properties, the physical property prediction model may be a classification model or a regression model, and the learning about the physical property prediction model is performed by applying substances with known physical properties and physical properties of the substances as inputs and outputs, respectively.

A computing apparatus for deriving a new drug candidate substance may include a knowledge network generating unit configured to generate a refined knowledge network in which nodes representing biological entities are connected by using a connecting line representing a correlation between the nodes, a basic drug determining unit configured to determine a basic drug for deriving a new drug candidate substance by analyzing drug-disease node pairs existing in the refined knowledge network, and an analogous substance acquiring unit configured to obtain an analogous substance having a chemical structure analogous to a structure of the basic drug by using an artificial neural network-based structure prediction model, wherein the biological entity includes at least one of a gene, a protein, a metabolite, a symptom, a disease, a compound, and a drug, the category of the correlation includes at least one of interact, participate, covariate, regulate, associate, bind, upregulate, cause, resemble, treat, downregulates, palliate, present, localize, include, and express, and a simplified molecular-input line-entry system (SMILES)-based character string of the basic drug is input in the structure prediction model.

The basic substance determining unit may calculate standard scores of proximities of drug-disease node pairs existing in the refined knowledge network, select at least one drug-disease node pair with the standard score of the proximity less than a reference value, and determine the drug of the selected at least one drug-disease node pair as the basic drug when a node indicating a disease exists on a path for the drug-disease node pair, and the standard score of the proximity may be a value obtained by dividing a difference between the shortest path of a specific pair of the drug-disease node pairs constituting the refined knowledge network and an average of shortest paths for the nodes constituting the refined knowledge network by a standard deviation.

The analogous substance obtaining unit may convert each of the characters constituting the SMILES-based character string for the basic drug into a vector of a reference size by replacing the character with an index corresponding to the character, extract a feature of the vector by encoding the vector, and determine a reconstruction vector generated by decoding the feature as the analogous substance.

The computing apparatus may include a physical property predicting unit configured to predict a physical property of the analogous substance through an artificial neural network-based physical property prediction model, wherein the physical property may include at least one of solubility, hydration energy, melting point, boiling point, toxicity, electrical stability, excited state property, protein-ligand binding, dissociation constant, and membrane permeability, the physical property prediction model may be independently generated for each of the physical properties, the physical property prediction model may be a classification model or a regression model, and the learning about the physical property prediction model may be performed by applying substances with known physical properties and physical properties of the substances as inputs and outputs, respectively.

### MODE FOR CARRYING OUT THE INVENTION

In the following, some embodiments will be described clearly and in detail with reference to the accompanying drawings so that those of ordinary skill in the art to which the present invention pertains (hereinafter, those skilled in the art) could easily implement the present invention.

In addition, the term “unit” used in the specification may mean a hardware component or circuit such as a field programmable gate array (FPGA) or an application specific integrated circuit (ASIC).

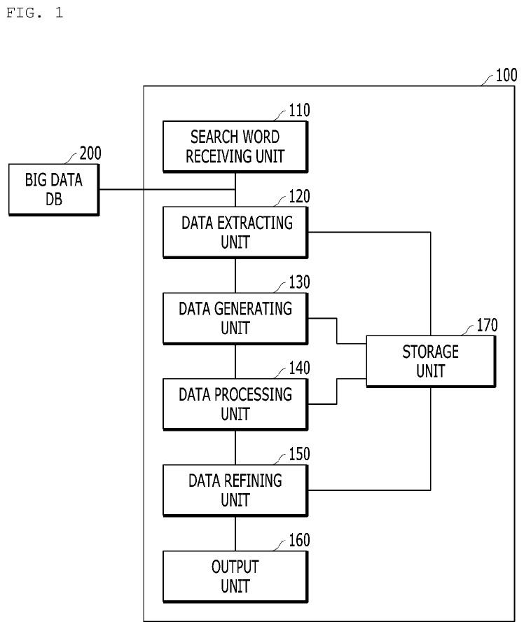

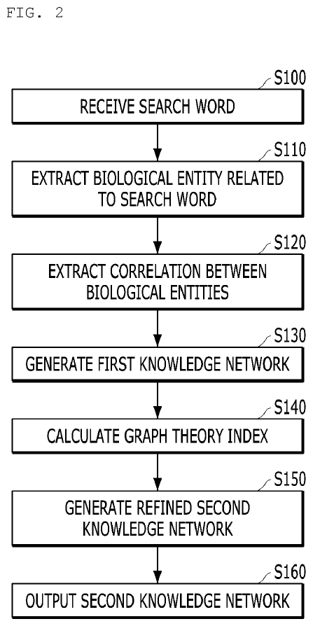

FIG. 1 is a block diagram of a data processing apparatus for discovering a new drug candidate substance according to an embodiment, and FIG. 2 is a flowchart illustrating a data processing method for discovering a new drug candidate substance by the data processing apparatus according to an embodiment.

Referring to FIG. 1, a data processing apparatus 100 for discovering a new drug candidate substance may include a search word receiving unit 110, a data extracting unit 120, a data generating unit 130, a data processing unit 140, and a data refining unit 150, an output unit 160, and a storage unit 170.

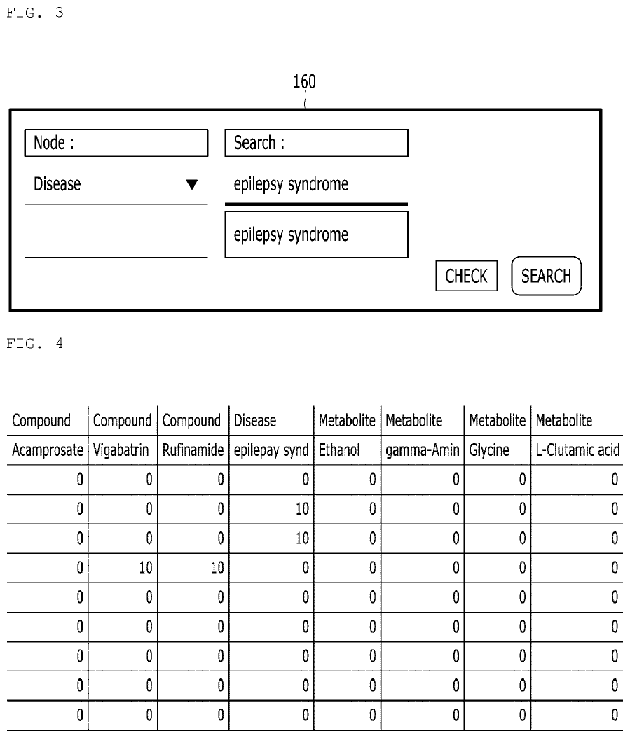

Referring to FIGS. 1 and 2, the search word receiving unit 110 may receive a predetermined search word (S100). The predetermined search word may be input through a user interface, and may include at least one of a gene name, a protein name, a metabolite name, a symptom name, a disease name, a compound name, and a drug name. For example, the user may input a drug called Bupropion as a search word or a disease called epilepsy syndrome as a search word, through the search word receiving unit 110. FIG. 3 illustrates an example in which a predetermined search word is input. Referring to FIG. 3, a screen for inputting a predetermined search word may be exposed through the output unit 160, and a predetermined search word may be input through a user interface. FIG. 3 illustrates an example of selecting a disease name as a category and inputting epilepsy syndrome as a predetermined search word.

Next, the data extracting unit 120 may extract at least one biological entity related to the predetermined search word received in step S100 (S110) , and may extract a correlation between the predetermined search word and the extracted biological entity (S120). Here, the biological entity may include at least one of a gene, a protein, a metabolite, a symptom, a disease, a compound, and a drug, and the level to which the predetermined search word belongs may be the same as or different from the level to which the biological entity belongs. For example, as illustrated in FIG. 3, when the predetermined search word is epilepsy syndrome which is a disease name, the biological entity extracted in step S110 may include a gene associated with epilepsy syndrome, a protein associated with epilepsy syndrome, and a metabolite associated with epilepsy syndrome, a symptom associated with epilepsy syndrome, a disease associated with epilepsy syndrome, a compound associated with epilepsy syndrome, and a drug associated with epilepsy syndrome. Further, for each level, a plurality of biological entities extracted in step S110 may be included. As illustrated in FIG. 3, when the predetermined search word is epilepsy syndrome which is a disease name, the biological entity extracted in step S110 may include a plurality of genes associated with epilepsy syndrome, a plurality of proteins associated with epilepsy syndrome, and a plurality of metabolites associated with epilepsy syndrome, a plurality of symptoms associated with epilepsy syndrome, a plurality of diseases associated with epilepsy syndrome, a plurality of compounds associated with epilepsy syndrome, and a plurality of drugs associated with epilepsy syndrome.

To this end, the data extracting unit 120 may use a big data DB 200. The big data DB 200 may be a database located outside or inside the data processing apparatus 100. The big data DB 200 is a database built for a public purpose, and anyone may access it or an authenticated person may access it under predetermined conditions. The big data DB 200 may store information on biological entities and correlations between biological entities in advance. For example, the big data DB 200 may include a DB for each type of biological entity and a DB for the correlation between biological entities.

The DB for each type of biological entity may include a gene DB, a protein DB, a metabolite DB, a symptom DB, a disease DB, a compound DB, and a drug DB. The DBs may be managed and operated by being integrated into one big data DB, or may be managed and operated by being distributed. The big data DB 200 may include an omics DB.

In order to extract at least one biological entity related to a predetermined search word and a correlation between biological entities, the data extracting unit 120 may use a natural language processing algorithm based on artificial intelligence technology including machine learning. Here, natural language processing refers to all kinds of technologies that mechanically analyze language phenomena spoken by humans and make them into a form that is able to be understood by a computer, and express the form that is able to be understood by a computer in a language that is able to be understood by humans. To this end, the big data DB 200 may be a language-based DB for each biological entity type, and may include information reflecting machine learning results and feedback results.

Alternatively, in order to extract at least one biological entity related to a predetermined search word and a correlation between biological entities, the data extracting unit 120 may be based on artificial intelligence technology including machine learning, and use a deep neural network algorithm. Here, the deep neural network is an artificial neural network composed of several hidden layers between the input layer and the output layer, and refers to various technologies used for classification, prediction, image recognition, and character recognition. To this end, the big data DB 200 may be an image-based DB for each biological entity type, and may include information reflecting machine learning results and feedback results.

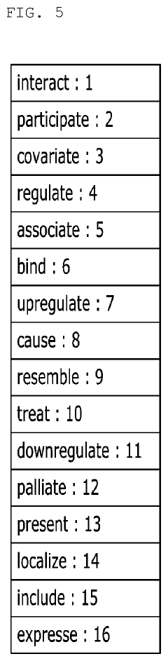

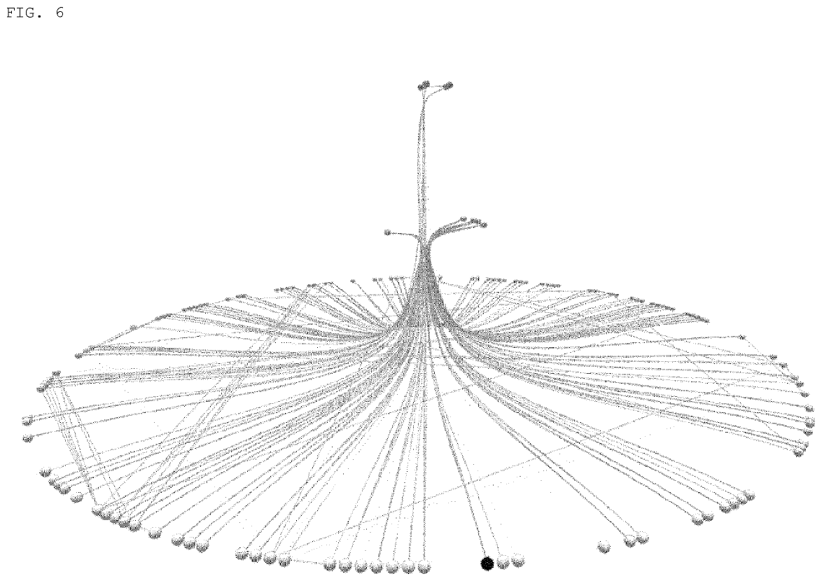

FIG. 4 is a part of an example of a matrix showing biological entities extracted in steps S110 and S120 and a correlation between the biological entities, and FIG. 5 is a part of an example of categories of correlations for extracting the matrix of FIG. 4.

Referring to FIGS. 4 and 5, the categories of the correlations between biological entities are “interact”, “participate”, “covariate”, “regulate”, and “associate”, “bind”, “upregulate”, “cause”, “resemble”, “treat”, “downregulates”, “palliate”, “present”, “localize”, “include”, “express”, or the like, and identification numbers may be randomly assigned by category. The identification number by category may be set by the user or may be set automatically.

For example, when the drug name bupropion is received as a predetermined search word in step S100, the data extracting unit 120 may extract “acamprosate”, “vigabatrin”, “rufinamide”, or the like as a compound related to bupropion, extract “epilepsy syndrome” as a disease, and extract “ethanol”, “gamma-amine”, “glycine”, “L-glutamic acid”, or the like as metabolites, and may generate a matrix in which categories of correlations between the predetermined search word and the biological entities or categories of correlations between biological entities are displayed as identification numbers. In the matrix of FIG. 4, the first column represents the categories of biological entities, the second column represents the biological entities extracted for each category, and the numbers in the lower column represent the categories of the correlations. The shape of the matrix is exemplary, and is not limited thereto, and may be modified into various shapes.

Next, the data generating unit 130 may generate a first knowledge network by using the results extracted in steps S110 and S120 (S130). FIG. 6 is an example of the first knowledge network generated according to an embodiment. A circle may represent a node, and a line may represent a connecting line (edge). Here, the first knowledge network may be in a form of a graph in which the predetermined search word received in step S100 and the biological entities extracted in step S110 are each set as a node and a plurality of nodes are connected to each other by using connecting lines according to the correlation between the extracted predetermined search word and the biological entities or the correlation between biological entities in step S120. Nodes within the same ohmic level may be connected through a connection line, or nodes within different ohmic levels may be connected through a connection line. Paths from a node A, which is one of the nodes in the first knowledge network, to a node B, which is the other, may vary, and all possible paths may be connected by connection lines. Here, the knowledge network is a network consisting of correlations between biological entities, and may also be referred to as a biological network.

Next, the data processing unit 140 calculates a graph theory index of the first knowledge network generated in step S130 (S140). According to an embodiment, the graph theory index may include at least one of a shortest inter-node path, a clustering coefficient per node, a centrality coefficient per node, and a hub characteristic by node for a plurality of nodes constituting the first knowledge network.

The shortest inter-node path may refer to the shortest path among numerous paths from the node A to the node B in the first knowledge network. Hereinafter, a method of calculating the shortest path between the node A, which is one of the biological entities, and the node B, which is the other one of the biological entities will be described.

There are various paths from the node A to the node B, and the node A and the node B may be directly connected, or at least one intermediate node may exist on each path between the node A and the node B. The data processing unit 140 may obtain the shortest path between the node A and the node B by using the number of intermediate nodes for each path. For example, the data processing unit 140 may determine that the path having a smaller number of intermediate nodes among various paths between the node A and the node B is a shorter path.

Alternatively, the data processing unit 140 may obtain the shortest path between the node A and the node B by using the number of intermediate nodes for each path, but may reflect the type of the correlation for each connecting line. That is, weights are set differently for each category of the correlation, and weights may be applied to correlations that exist for each path. The types of correlations are as illustrated in FIG. 5 and may have different weight values for each type of correlation.

Equation 1 is an example of an equation that calculates the shortest path between nodes.

$${d}_{i , j}^{w} \text{?} f ( {w}_{st} ) \\ \text{?} \text{indicates text missing or illegible when filed} \qquad [ Equation 1 ]$$

where wₛₜ is the correlation index between two nodes s and t, f is a weight transformation function, g_{i→j}^{w} is the shortest path between two nodes i and j. The data processing unit 140 may determine the value of Equation 1 for each path, and may select a path having the lowest value or the highest value as the shortest path.

Next, a clustering coefficient per node may be calculated by Equation 2 and Equation 3. Here, the clustering coefficient may be referred to as a clustering coefficient, and may refer to a probability that a specific node and neighboring nodes are connected to each other or a connection density between a specific node and neighboring nodes.

$${t}_{i}^{w} = \frac{1}{2} {\sum }_{j , h ∈ N} {w}_{ij} {w}_{ih} {w}_{jh} \qquad [ Equation 2 ]$$

where, tᵢ^{w} is the number of triangles in the graph created around each node i of the knowledge network, N is the total node set of the knowledge network, and wᵢⱼ is the correlation index between the node i and the node j, wᵢₙ is the correlation index between the node i and a node h, and wⱼₕ is a correlation index between the node j and the node h.

$${C}^{w} = \frac{1}{n} {\sum }_{i ∈ N} \frac{2 {t}_{i}^{w}}{{k}_{i} ( {k}_{i} - 1 )} \qquad [ Equation 3 ]$$

where, C^{w} is the clustering coefficient, tᵢ^{w} is the number of triangles in the graph created around each node i of the knowledge network, and kᵢ is the degree of the node i, that is, a value of the degree of connectivity of the node i in the knowledge network.

Next, a centrality index per node is an index for whether a specific node has the function of a hub, and may be expressed as a nodal degree (D_{nodal}) value, a betweenness centrality (BC) value, a nodal efficiency (E_{nodal}) value. Here, the value of D_{nodal} is a value of the degree of connectivity of each node in the knowledge network, that is, an index indicating how strong or weak a node i has connectivity in the knowledge network, the value of E_{nodal} is a value of the degree of efficiency of the node i in the knowledge network, that is, a value expressed by the reciprocal of the shortest path in Equation 1 and the shorter the path, the higher the efficiency, and the BC value is an index indicating the number of times the node i becomes a shortcut in the path between nodes in the knowledge network.

First, the value of D_{nodal} may be calculated by

Equation 4.

$${D}_{nodal} ( i ) = {\sum }_{j ∈ N} {w}_{ij} \qquad [ Equation 4 ]$$

where, wᵢⱼ is the correlation index between the node i and the node j, and N is the total node set of the knowledge network.

Then, the value of E_{nodal} may be calculated by

Equation 5.

$${E}_{nodal ( i )} = {\sum }_{j ∈ N , j \neq i} \frac{1}{{d}_{i , j}^{w}} \qquad [ Equation 5 ]$$

where, N is a set of all nodes of the knowledge network, and is a value representing the shortest path calculated in Equation 1.

Next, the Betweenness centrality (BC) may be calculated by Equation 6.

$$BC ( i ) = {\sum }_{{h , j ∈ N}_{h \neq j , h \neq i , j \neq i}} \frac{{g}_{hj} ( i )}{{g}_{hj}} \qquad [ Equation 6 ]$$

where, gₕⱼ refers to the shortest distance between the node h and the node j, and gₕⱼ(i) refers to the shortest distance between the node h and the node j passing through the node i.

Next, when it is determined that the predetermined node has the function of a hub, the data processing unit 140 may classify the nature of the hub. In this case, the nature of the hub may be classified into a kinless hub, a connector hub, a provincial hub, and the like. Here, the kinless hub refers to a hub that has the highest influence, that is, a hub connected to many in-module nodes, the connector hub refers to a hub that connects modules in the knowledge network, and the provincial hub refers to a hub that mainly has a high influence within a module. Here, the module may be a structural configuration group obtained by subdividing the entire knowledge network.

To this end, a module index (modularity) in the knowledge network may be calculated as in Equation 7. The Modularity refers to the number of types of configuration modules in the entire knowledge network.

$${Q}^{w} = \frac{1}{{l}^{w}} {\sum }_{i , j ∈ N} [ {w}_{ij} - \frac{{k}_{i}^{w} {k}_{j}^{w}}{{l}^{w}} ] {σ}_{mi , mj} \qquad [ Equation 7 ]$$

where, kᵢ^{w}=Σ_{j∈N}wᵢⱼ is the sum of weights at the node i, and i^{w}=Σ_{i,j∈N}^{w}^{ᵢⱼ} is the sum of weights. δ_{mi,mj} is the kronecker delta, which is 1 for mi=mj, and 0 for the remainder.

Next, a participation coefficient (PC) of the knowledge network module may be calculated as in Equation 8.

$${PC}_{i} = 1 - {\sum }_{m ∈ M} {[ \frac{{k}_{i}^{w} ( m )}{{k}_{i}^{w}} ]}^{2} \qquad [ Equation 8 ]$$

where, M is a set of modules, kᵢ^{w}(m) is the number of connections between a node i and all other nodes in a module m, and the module m is a structural group obtained by subdividing entire knowledge network.

Then, the z score (within-module degree) of the knowledge network module may be calculated as in Equation 9.

$${z}_{i}^{w} = \frac{{k}_{i}^{w} ( {m}_{i} ) - {\overline{k}}^{w} ( {m}_{i} )}{{σ}_{k}^{w} ( {m}_{i} )} \qquad [ Equation 9 ]$$

where, mᵢ is a node i in a module m, kᵢ^{w}(mᵢ) is the degree of connection within the module m of the node i, and k̅^{w}(mᵢ), σₖ^{w} (mᵢ) is the mean and standard deviation of the degree distribution of connection in the module m, respectively.

It is possible to distinguish whether or not each node is a hub in the module through the calculation of the index of Equation 9 above. For example, as follows, if the Z score of the knowledge network module is 2.5 or higher, it may be determined that the node is the hub.

1. within-module z-score ≥2.5: hub

2. within-module z-score <2.5: not hub

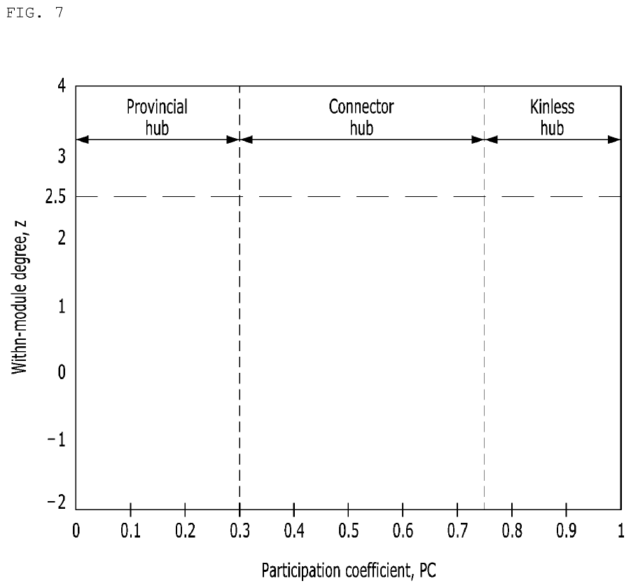

In addition, when it is determined that the node is a hub in the module, the type of the hub may be classified as follows through the calculation of the index of Equation 8, and FIG. 7 illustrates an example of classifying the type of the hub according to the PC.

1. Provincial hub: PC≤0.30

2. Connector hub: 0.3<PC≤0.75

3. Kinless hub: PC>0.75

As described above, when the data processing unit 140 calculates the graph theory index in step S140, the data refining unit 150 generates a second knowledge network refined from the first knowledge network by using the graph theory index (S150). The second knowledge network is a network that is more simplified than the first knowledge network, and may be composed of only some nodes having the high correlation in terms of graph theory among a plurality of nodes constituting the first knowledge network.

Nodes constituting the second knowledge network may be composed of nodes, of a plurality of nodes constituting the first knowledge network, of which the graph theory index calculated in step S140 is equal to or greater than a reference value. For example, among a plurality of nodes constituting the first knowledge network, some nodes of which at least some of the index values for the shortest inter-node path, the clustering coefficient per node, and the centrality coefficient per node are equal to or greater than a reference value may be included in the second knowledge network. In other words, the second knowledge network may be generated in a manner in which, among the plurality of nodes constituting the first knowledge network, nodes of which at least some of the index values for the shortest inter-node path, the clustering coefficient per node, and the centrality coefficient per node are less than a threshold value are deleted and connections associated with the deleted nodes are deleted.

Here, the graph theory index, which is compared with the reference value, may be index values for the shortest inter-node path, an index value for the clustering coefficient per node, or an index value for the centrality coefficient per node. Alternatively, the graph theory index compared with the reference value may be a value calculated by integrating at least two of the index value for the shortest inter-node path, the index value for the clustering coefficient per node, and the index value for the centrality coefficient per node.

According to an embodiment, at least one of the index value for the shortest inter-node path, the index value for the clustering coefficient per node, and the index value for the centrality coefficient per node may be calculated as a standard score for each node, and the calculated standard score may be compared with the threshold value.

Here, the standard score may be a z score, and the threshold value may refer to 95% significance. The z score may be calculated as in Equation 10.

$$z = \frac{X - mean ( x )}{SE ( x )} \qquad [ Equation 10 ]$$

where z is the z score, X is index values of a predetermined graph theory index for a specific node in the first knowledge network, and mean(x) is an average index value of a predetermined graph theory index for at least some nodes in the first knowledge network, and SE(x) is a standard error of the index values of the graph theory index for at least some nodes in the first knowledge network. Here, SE=σ/\sqrt{N}, where o is the standard deviation, and n is the number of at least some nodes constituting the first knowledge network. According to an embodiment, the number of at least some nodes of the first knowledge network selected to determine the z score may be 1000.

That is, the z score may be a value obtained by dividing a difference between an index value of a predetermined graph theory index for each of the nodes constituting the first knowledge network and an average index value of a predetermined graph theory index for the plurality of nodes constituting the first knowledge network by a standard error.

According to an embodiment, the z score may be calculated through a permutation test. The permutation test may be performed by randomly mixing all connecting lines constituting the first knowledge network, and then calculating a z score for each node. In this case, the number of random mixing may be 1000 times or more.

The nodes constituting the second knowledge network may be some nodes extracted among the plurality of nodes constituting the first knowledge network by using the index value for the nature of the hub by node in the graph theory index calculated in step S140. That is, the nodes constituting the second knowledge network are nodes determined as the in-module hub through the calculation of the index of Equation 9, preferably a node classified as one of a kinless hub, a connector hub, and a provincial hub, more preferably a node classified as one of a kinless hub and a connector hub, and even more preferably a node classified as a kinless hub.

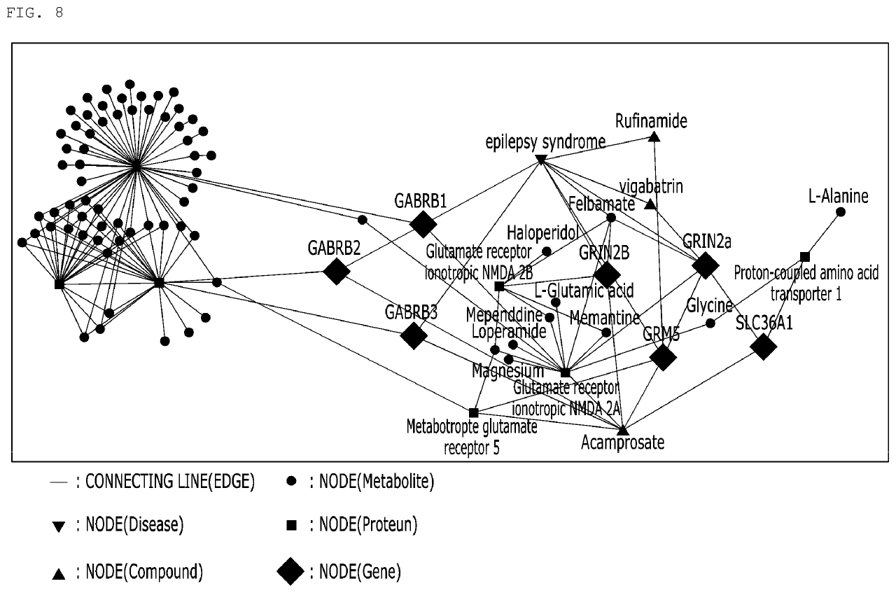

Next, the output unit 160 outputs the second knowledge network generated in step S150 (S160). The output unit 160 may be, for example, a display. FIG. 8 is an example of the second knowledge network generated by using “epilepsy syndrome” as a search word, according to an embodiment of the present invention. Referring to FIG. 8, it can be seen that a significantly simplified and refined second knowledge network may be obtained, compared to the first knowledge network of FIG. 6. In addition, referring to FIG. 8, it can be seen that biological entities within different ohmic levels associated with “epilepsy syndrome” and the correlation between the biological entities may be intuitively obtained.

In this way, the data processing apparatus 100 may generate the second knowledge network composed of only nodes refined in relation to a predetermined search word, and accordingly, may easily determine a new drug candidate substance or a target of a new drug candidate substance.

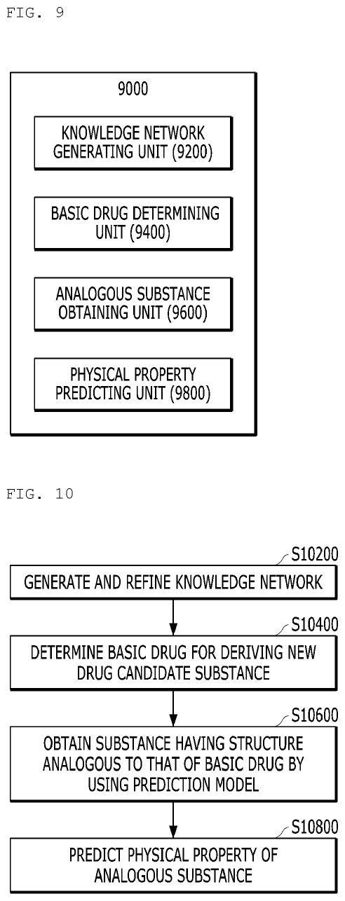

FIG. 9 is a block diagram of a computing apparatus for deriving a new drug candidate substance, according to an embodiment.

A computing apparatus 9000 may include at least one processor (not illustrated) and at least one memory (not illustrated). The processor may include a central processing unit (CPU), a microprocessor, a graphic processing unit (GPU), a digital signal processor (DSP), or a micro controller unit (MCU).

The memory may include volatile memory such as dynamic random access memory (DRAM) and static random access memory (SRAM), and non-volatile memory such as flash memory, read only memory (ROM), phase-change random access memory (PRAM), magnetic random access memory (MRAM), resistive random access memory (ReRAM), and ferroelectrics random access memory (FRAM).

Referring to FIG. 9, the computing apparatus 9000 may include a knowledge network generating unit 9200, a basic drug determining unit 9400, an analogous substance obtaining unit 9600, and a physical property predicting unit 9800. Each of the knowledge network generating unit 9200, the basic drug determining unit 9400, the analogous substance obtaining unit 9600, and the physical property predicting unit 9800 may be hardware components or circuits, and may include at least one processor and/or at least one memory.

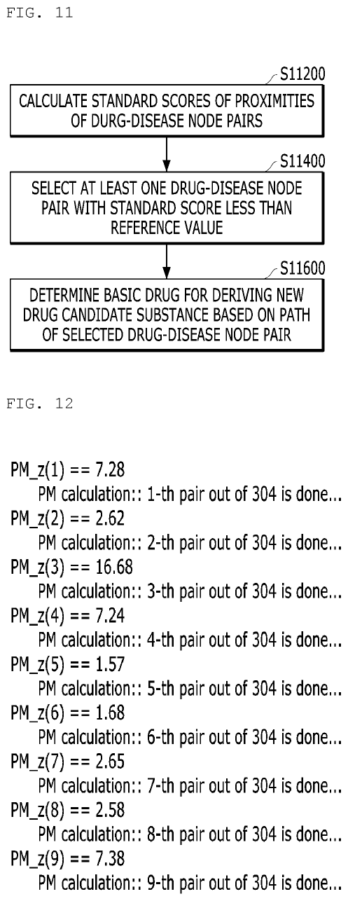

Referring to FIGS. 9 and 10 together, the computing apparatus 9000 may generate and refine the knowledge network in step S10200. According to an embodiment, step S10200 may be performed by the knowledge network generating unit 9200 of the computing apparatus 9000.

The computing apparatus 9000 may generate a refined knowledge network in which nodes representing biological entities are connected by using a connecting line (or edge) representing a correlation between the nodes. For example, the biological entity may include at least one of a gene, a protein, a metabolite, a symptom, a disease, a compound, and a drug. For example, the category of the correlation may include at least one of interact, participate, covariate, regulate, associate, bind, upregulate, cause, resemble, treat, downregulates, palliate, present, localize, include, and express.

The refined knowledge network generated in step S10200 may be a knowledge network such as the second knowledge network described above with reference to FIGS. 1 to 8. According to an embodiment, the knowledge network generating unit 9200 may include the data processing apparatus 100 of FIG. 1. A method of generating the refined knowledge network by the knowledge network generating unit 9200 is the same as described above with reference to FIGS. 1 to 8, and thus a detailed description thereof will not be repeated.

The computing apparatus 9000 may determine a basic drug for deriving a new drug candidate substance in step S10400. The computing apparatus 9000 may determine a basic drug for deriving a new drug candidate substance by analyzing drug-disease node pairs existing in the refined knowledge network generated in step S10200. The computing apparatus 9000 may determine a basic drug based on the proximity of the drug-disease node pair. Step S10400 may be performed by the basic drug determining unit 9400 of the computing apparatus 9000.

FIG. 11 is a flowchart of a method for determining a basic drug by a computing apparatus, according to an embodiment. The flowchart of FIG. 11 may represent sub-steps of step S10400 of FIG. 10.

Referring to FIG. 11, in step S11200, the computing apparatus 9000 may calculate a standard score (z-score) of the proximity of each of drug-disease node pairs existing in the refined knowledge network. The computing apparatus 9000 may determine, from the knowledge network, at least one drug-disease node pair in which a specific drug node and a disease node connected with the specific drug node through a connecting line are set as a source node and a target node, respectively. According to an embodiment, the computing apparatus 9000 may extract all drug-disease pairs for a specific drug from the knowledge network, and may calculate a standard score of the proximity of each of the extracted drug-disease pairs. According to an embodiment, the standard score of the proximity of the node pair (s, t) (s: source node (drug), t: target node (disease)) may be calculated by using Equation 11 below.

$$z ( s , t ) = \frac{d ( s , t ) - mean ( d ( s , T ) )}{SD ( d ( s , T ) )} \qquad [ Equation 11 ]$$

(s: source node, t: current target node, T: set of target nodes, d(s, t): shortest path (shortest distance) between the source node s and the current target node t, mean(d(s, T)): the mean of the shortest paths for node pairs consisting of the source node s and the target node set T, SD(d(s, T)): the standard deviation of the shortest paths for node pairs consisting of the source node s and the target node set T, and z(s, t): the standard score (z-score) of the proximity of the source node s and the current target node t).

According to an embodiment, the set of target nodes used to determine the mean and the standard deviation may be disease nodes of drug-disease pairs extracted from the refined knowledge network. However, the set of target nodes is not limited to disease nodes. For example, the set of target nodes used to determine the mean and standard deviation may be nodes randomly selected from the refined knowledge network. The computing apparatus 9000 may determine N (N is a positive integer) drug-target node pairs from the knowledge network (here, the biological entity of the target node is a gene, a protein, a metabolite, a symptom, a disease, a compound, a drug, or the like), and the determined N node pairs may be used as the target node set for calculating a standard score of proximity. N may be the number of sample data (for example, 1000 or more) that are expected to be statistically normal distribution.

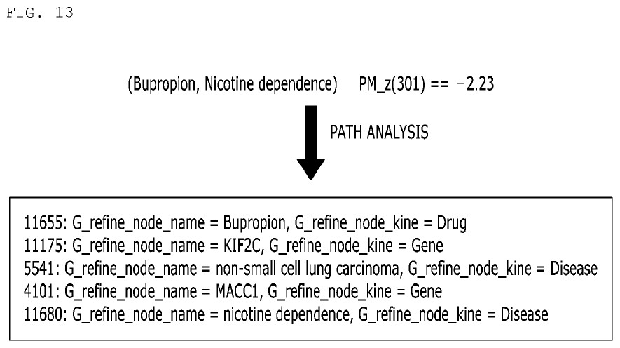

Referring to FIG. 12, the computing apparatus 9000 may extract K drug-disease node pairs (K is a positive integer) with a node representing Bupropion as a source node and nodes representing diseases as target nodes. For example, the computing apparatus 9000 may extract 304 bupropion-disease node pairs and determine a standard score of the proximity of each of the extracted node pairs. For example, the standard score of the first node pair may be determined to be 7.28, the standard score of the second node pair may be determined to be 2.62, and the standard score of the ninth node pair may be determined to be 7.38.

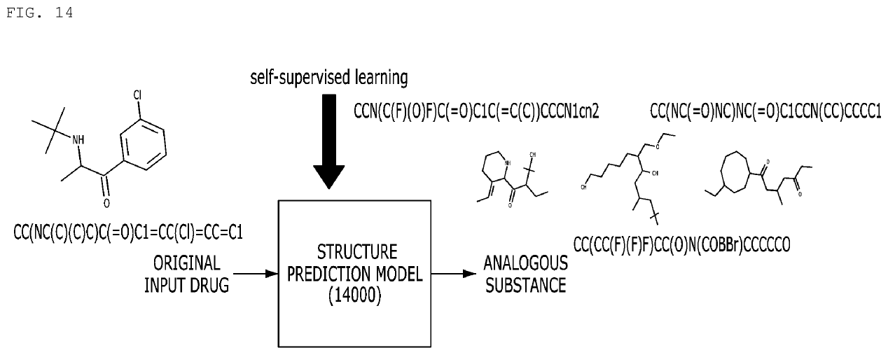

Referring back to FIG. 11, in step S11400, the computing apparatus 9000 may select, from among drug-disease node pairs, at least one drug-disease node pair having a standard score of proximity less than a reference value. For example, when the reliability is set to 90%, the reference value may be determined to be −1.645, when the reliability is set to 95%, the reference value may be determined to be −1.960, and when the reliability is set to 99%, the reference value may be determined to be −2.576. Referring to FIG. 13, when the reliability is set to 95%, the computing apparatus 9000 may select the 301-th node pair from among 304 drug-disease node pairs. The source node and target node of the 301-th node pair are Bupropion and Nicotine dependence, respectively, and the standard score of the proximity is −2.23, which is less than the standard value of −1.960.

Referring back to FIG. 11, in step S11600, the computing apparatus 9000 may determine a basic drug for deriving a new drug candidate substance based on the path of the selected at least one drug-disease node pair. If there is an intermediate node indicating a disease on the path from the source node s to the target node t in the drug-disease node pair selected in step S11400, the computing apparatus 9000 may determine the source node s of the drug-disease node pair, that is, the drug as a basic drug for deriving a new drug candidate substance.

Referring back to FIG. 13, the source node and the target node in the selected drug-disease node pair (Bupropion, Nicotine dependence) are a node 11655 (Bupropion) and a node 11680 (Nicotine dependence), respectively, and on the path from the source node to the target node, nodes 11175, 5541, and 4101 exist.

The name of the source node (identification number: 11655) is Bupropion and the biological entity type is Drug. The name of the intermediate node (identification number: 11175) is KIF2C and the biological entity type is Gene. The name of the intermediate node (identification number: 5541) is non-small cell lung carcinoma and the biological entity type is Disease. The name of the intermediate node (identification number: 4101) is MACC1 and the biological entity type is Gene. The name of the target node (identification number: 11680) is nicotine dependence, and the biological entity type is Disease.

Referring to the path of the selected drug-disease node pair (Bupropion, Nicotine dependence), in addition to the direct correlation between bupropion and nicotine dependence, the correlation between bupropion and non-small cell lung carcinoma may be derived. In addition to nicotine dependence, an existing indication for bupropion, a new indication, non-small cell lung carcinoma, may be discovered based on the path of the selected drug-disease node pair. The computing apparatus 9000 may determine bupropion as a basic drug for deriving a new drug candidate substance because a node representing a disease exists on the path of the selected drug-disease node pair (Bupropion, Nicotine dependence). That is, the computing apparatus 9000 may determine a drug from which a new indication may be derived by analyzing the path of the drug-disease node pair selected based on the proximity of the drug-disease node pairs, and may determine the determined drug as a basic drug for deriving a new drug candidate substance.

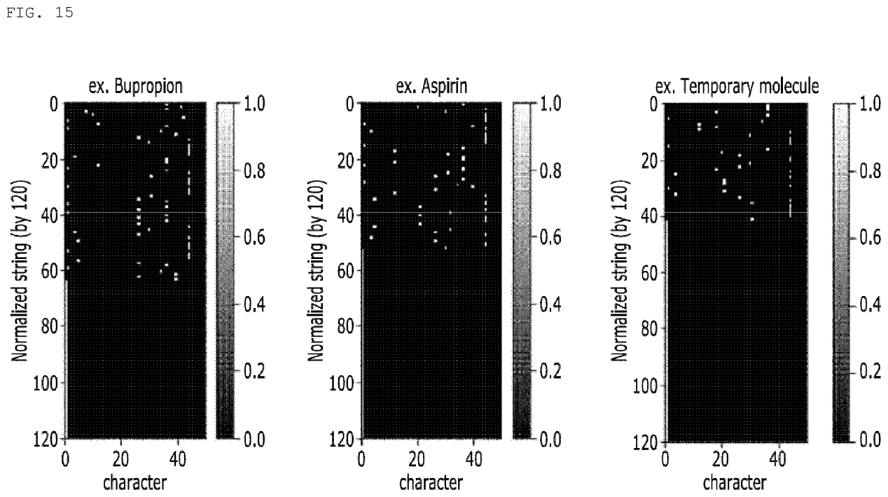

Referring back to FIG. 10, in step S10600, the computing apparatus 9000 may obtain an analogous substance having a chemical structure analogous to that of the determined basic drug by using a structure prediction model based on an artificial neural network. Referring to FIG. 14, the computing apparatus 9000 may input the basic drug (for example, bupropion) to a structure prediction model 14000, and may obtain, from the structure prediction model 14000, a substance having a structure chemically analogous to the basic drug. Since the pharmacological mechanisms of the drug having the chemical structure analogous to that of the basic drug are expected to be analogous to those of the basic drug, it means that the drug having the chemical structure analogous to that of the basic drugs may be the candidate substance for the new drug. Step S10600 may be performed by the analogous substance obtaining unit 9600 of the computing apparatus 9000. The analogous substance obtaining unit 9600 may include the structure prediction model 14000.

The structure prediction model 14000 may be generated based on an artificial intelligence algorithm. According to an embodiment, the structure prediction model 14000 may be based on an artificial neural network including an input layer, a hidden layer, and an output layer. The artificial neural network may be stored in a memory of the computing apparatus 9000 (for example, the analogous substance obtaining unit 9000). Each of the input layer, the hidden layer, and the output layer may include a plurality of neurons, and the neurons may be connected to synapses having weights. Hereinafter, the structure prediction model 14000 may refer to an artificial neural network.

According to an embodiment, learning about the structure prediction model 14000 may be performed based on self-supervised learning, such as a variational autoencoder (VAN) or a generative adversarial network (GAN). In the embodiment, learning about the structure prediction model 14000 may be performed to output the same data as input data.

For example, the artificial neural network of the structure prediction model 14000 may encode input data to extract features, and may generate reconstructed data by decoding the extracted features. In the embodiment, for the artificial neural network of the structure prediction model 14000, the number of neurons in the input layer and the number of neurons in the output layer may be the same, and the number of neurons in the hidden layer may be less than that of neurons in the input layer. In the artificial neural network, the flow from the input layer to the hidden layer is an encoding process, and the process from the hidden layer to the output layer is a decoding process. Learning about the artificial neural network of the structure prediction model 14000 may be performed so that the input and the output have the same value.

The artificial neural network of the structure prediction model 14000 may extract features by encoding the input basic drug, and may generate reconstructed data by decoding the extracted features. The reconstructed data may be an analogous substance to be obtained. The loss representing the performance of the artificial neural network may be determined to be smaller as the output substance has a structure analogous to the chemical structure of the input basic drug, and it may be determined that the smaller the loss, the better the performance of the artificial neural network. Learning about the artificial neural network may be performed by using a back propagation algorithm for updating synaptic weights to reduce the loss between the input and the current output corresponding to the input.

According to an embodiment, the input and output of the structure prediction model 14000 may be character strings expressed according to the simplified molecular-input line-entry system (SMILES) of a substance. Referring to FIG. 14, when a character string CC(NC(C (C)C)C(═O)C1═CC(C1)═CC═C1 representing bupropion is input to the structure prediction model 14000, CCN(C(F)(O)F)C(═O)C1C(═C(C))CCCN1cn2, CC(CC(F) (F)F)CC(O)N(COBBr)CCCCCO, and CC(NC(═O)NC)NC(═O)C1CCN(CC)CCC1, which have a chemical structure analogous to bupropion, may be output. Alternatively, when CC(═O)OC1═CC═CC═C1C(O)═O representing aspirin is input into the structure prediction model 14000, OC (═O)OC1═CCCC═CC1nF, CC(═N)NC1═CN═CCCCC1n1, and CC(═O)CN1CSSCOOC(NP(═O) (O)O)n1, which have a chemical structure analogous to aspirin may be output.

According to an embodiment, in step S10600, the computing apparatus 9000 mal normalize and convert the SMILES-based character string into a vector of the reference size so that the structure prediction model 14000 is able to easily understand the input data and facilitate feature extraction from the input data. The computing apparatus 9000 may normalize and convert all SMILES-based character strings into vectors of the same size (for example, 120) according to a preset reference size value. The computing apparatus 9000 may convert the SMILES-based character string for the basic drug into a vector having the reference size by replacing each of the characters constituting the SMILES-based character string with a number (or index) corresponding to the character. As an example, the index may be a position in a character set composed of characters constituting the SMILES-based character string.

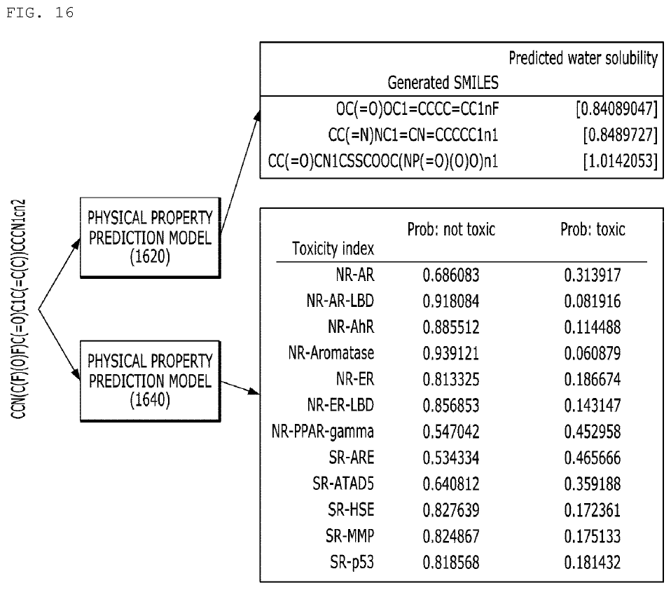

FIG. 15 is a diagram illustrating that a basic drug input to a prediction model is transformed into a vector of a reference size, according to an embodiment. Referring to FIG. 15, bupropion may be assigned an index value corresponding to a character from the first position to the 64-th position, and may be converted into a vector of size 120 padded with 0 from the 65-th position to the 120-th position. In addition, aspirin may be assigned an index value corresponding to a character from the first position to the 54-th position, and may be converted into a vector of size 120 padded with 0 from the 55-th position to the 120-th position. In addition, a temporary molecule may be assigned an index value corresponding to a character from the first position to the 42-th position, and may be converted into a vector of size 120 padded with 0 from the 42-th position to the 120-th position.

According to an embodiment, in step S10600, the structure prediction model 14000 may extract features of the vector by encoding the vector of the reference size, and may output a reconstructed vector for the vector of the reference size by decoding the extracted features. The reconstructed data may represent an analogous substance to be obtained.

In step S10800, the computing apparatus 9000 may predict the physical properties of the analogous substance. Physical properties may include physicochemical properties such as water solubility, hydration energy, melting point, and boiling point, physiological properties such as toxicity, quantum mechanical properties such as stability based on electronic properties and excited state properties (QM8), and biophysical properties such as protein-ligand binding, dissociation constant, and membrane permeability. Step S10800 may be performed by a physical property predicting unit 9800 of the computing apparatus 9000.

The computing apparatus 9000 may utilize a physical property prediction model based on the artificial neural network for physical property prediction. The physical property prediction model may be separate from the structure prediction model 14000 of FIG. 14 and may be independently generated for each of the physical properties. For example, referring to FIG. 16, the computing apparatus 9000 may store a physical property prediction model 1620 for predicting solubility and a physical property prediction model 1640 for predicting toxicity in memory, respectively.

According to an embodiment, the physical property prediction model may be a classification model or a regression model. For example, the physical property prediction model 1620 may output the solubility of an analogous substance as a numerical value by using the regression model. The physical property prediction model 1640 may output toxicity of an analogous substance as a probability of existence of toxicity by using the classification model. When the probability of the existence of toxicity is equal to or greater than a reference value, the analogous substance may be determined as being toxic.

The learning about the physical property prediction model may be performed by applying substances with known physical properties and physical properties of the substances as inputs and outputs, respectively. For example, the loss representing the performance of the physical property prediction model 1620 for predicting solubility may be determined as the difference between the output solubility and the known solubility of the input substance, and it may be determined that the smaller the loss, the better the performance of the physical property prediction model 1620. In addition, the loss representing the performance of the physical property prediction model 1640 for predicting toxicity may mean whether or not the output toxicity is consistent with the known toxicity of the input substance, and it may be determined that the smaller the loss, the better the performance of the physical property prediction model 1640.

Learning about the physical property prediction model (for example, physical property prediction model 1620 or physical property prediction model 1640) may be performed by using a back propagation algorithm for updating synaptic weights to reduce the loss between the input and the current output corresponding to the input.

Meanwhile, the method for deriving a new drug candidate substance described above may be implemented as a computer-readable code on a computer-readable recording medium. The computer-readable recording medium includes all types of recording devices storing data that is readable by a computer system. Examples of the computer-readable recording media include a read only memory (ROM), a random access memory (RAM), a compact disc (CD)-ROM, magnetic tapes, floppy disks, optical data storage devices, and the like, and also include those implemented in the form of transmission through the Internet. In addition, the computer-readable recording medium is distributed over a computer system connected through a network, where the code that is readable by the processor may be stored and executed in a distributed manner.

The descriptions are intended to provide exemplary configurations and operations for implementing the present invention. The technical idea of the present invention is to include not only the embodiments described above, but also implementations that may be obtained by simply changing or modifying the above embodiments. In addition, the technical idea of the present invention is also to include implementations that may be achieved by easily changing or modifying the embodiments described above.

1. A method for deriving a new drug candidate substance that is executed by a computing apparatus, the method comprising:
generating a refined knowledge network in which nodes representing biological entities are connected to each other by using a connecting line representing a correlation between the nodes based on a database (DB) for each biological entity type and a DB for a correlation between biological entities;
determining a basic drug for deriving a new drug candidate substance by analyzing drug-disease node pairs existing in the refined knowledge network;
obtaining an analogous substance having a chemical structure analogous to a structure of the basic drug by using an artificial neural network-based structure prediction model, and
predicting a physical property of the analogous substance through an artificial neural network-based physical property prediction model,
wherein the biological entity includes at least one of a gene, a protein, a metabolite, a symptom, a disease, a compound, and a drug,
a category of the correlation includes at least one of interact, participate, covariate, regulate, associate, bind, upregulate, cause, resemble, treat, downregulates, palliate, present, localize, include, and express,
wherein the determining of the basic drug for deriving the new drug candidate substance includes:
calculating standard scores of proximities of the drug-disease node pairs existing on the refined knowledge network;
selecting at least one drug-disease node pair with the standard score of the proximity less than a reference value; and
determining the drug indicated by a source node of the selected at least one drug-disease node pair as the basic drug when a intermediate node indicating a disease different from a disease indicated by a target node exists on a path for the drug-disease node pair,
a simplified molecular-input line-entry system (SMILES)-based character string of the basic drug is input in the structure prediction model, and
wherein the physical property includes at least one of solubility, hydration energy, melting point, boiling point, toxicity, electrical stability, excited state property, protein-ligand binding, dissociation constant, and membrane permeability.

2. The method of claim 1, wherein the generating of the refined knowledge network includes:
receiving a search word;
extracting at least one biological entity related to the search word from the database (DB) for each biological entity type;
extracting a correlation between the search word and the biological entities from the DB for a correlation between biological entities;
generating a first knowledge network in which the search word and the biological entities are each set as a node and a plurality of nodes are connected to each other by using a connecting line according to the correlation between the search word and the biological entities or the correlation between the biological entities;
calculating a graph theory index of the first knowledge network; and
generating a second knowledge network as the refined knowledge network by using a portion of the plurality of nodes that are extracted by using the graph theory index,
the search word includes at least one of a gene name, a protein name, a metabolic name, a symptom name, a disease name, a compound name, and a drug name,
an identification number is assigned and a weight is set for each category of the correlation, and the graph theory index is calculated by reflecting the weight set for each category of the correlation,
the graph theory index includes at least one of a shortest inter-node path, a clustering coefficient per node, a centrality coefficient per node, and a nature of a hub by node for the plurality of nodes constituting the first knowledge network, and
the generating of the second knowledge network includes:
calculating a standard score per node by using at least one of the shortest inter-node path, the clustering coefficient per node, and the centrality coefficient per node for the plurality of nodes constituting the first knowledge network among the plurality of nodes, deleting a node of which the standard score is less than a threshold value, and deleting the connection associated with the deleted node.

3. (canceled)

4. The method of claim 1, wherein the
standard scores of proximities of the drug-disease node pairs is calculated via the following <Equation>
$$\underline{z ( s , t ) = \frac{d ( s , t ) - mean ( d ( s , T ) )}{SD ( d ( s , T ) )}} \qquad \underline{\text{<} Equation \text{>}}$$
wherein the s is a source node indicating drug, the t is a target node indicating disease, z(s, t)is the standard scores of proximities of the source node s and the target node t, d(s, t) is the shortest path between the source node s and the target node t, the T is a set of target nodes, the mean(d(s,T)) is the mean of the shortest paths for node pairs consisting of the source node s and the target node ser T, and the SD(d(s, T)) is the standard deviation of the shortest paths for node pairs consisting of the source node s and the target node set T, the set of target nodes may be nodes randomly selected from the refined knowledge network.

5. (canceled)

6. The method of claim 1, wherein the obtaining of the analogous substance having the chemical structure analogous to the structure of the basic drug includes:
converting each of characters constituting the SMILES-based character string for the basic drug into a vector of a reference size by replacing the character with an index corresponding to the character; and
determining an output obtained by inputting the vector into the structure prediction model as the analogous substance.

7. The method of claim 6, wherein the determining of the output obtained by inputting the vector into the structure prediction model as the analogous substance includes:
extracting a feature of the vector by encoding the vector; and
outputting a reconstruction vector by decoding the feature.

8. The method of claim 7, wherein the artificial neural network includes
an input layer, a hidden layer, and an output layer,
the number of neurons in the input layer and the output layer is the same, and the number of neurons in the hidden layer is less than the number of neurons in the input layer.

9. The method of claim 1, wherein learning about the structure prediction model is performed based on self-supervised learning in which a synapse of the artificial neural network is updated to generate the same output as the input to the structure prediction model.

10. (canceled)

11. The method of claim 1, wherein the physical property prediction model is independently generated for each of the physical properties,
the physical property prediction model is a classification model or a regression model, and
the learning about the physical property prediction model is performed by applying substances with known physical properties and physical properties of the substances as inputs and outputs, respectively.

12. A computing apparatus for deriving a new drug candidate substance, the computing apparatus comprising:
a knowledge network generating unit configured to generate a refined knowledge network in which nodes representing biological entities are connected by using a connecting line representing a correlation between the nodes based on a database (DB) for each biological entity type and a DB for a correlation between biological entities;
a basic drug determining unit configured to determine a basic drug for deriving a new drug candidate substance by analyzing drug-disease node pairs existing in the refined knowledge network;
an analogous substance acquiring unit configured to obtain an analogous substance having a chemical structure analogous to a structure of the basic drug by using an artificial neural network-based structure prediction model; and
a physical property predicting unit configured to predict a physical property of the analogous substance through an artificial neural network-based physical property prediction model, wherein
the biological entity includes at least one of a gene, a protein, a metabolite, a symptom, a disease, a compound, and a drug,
a category of the correlation includes at least one of interact, participate, covariate, regulate, associate, bind, upregulate, cause, resemble, treat, downregulates, palliate, present, localize, include, and express, and
the basic drug determining unit calculates standard scores of proximities of drug-disease node pairs existing in the refined knowledge network, selects at least one drug-disease node pair with the standard score of the proximity less than a reference value, and determines the drug indicated by a source node of the selected at least one drug-disease node pair as the basic drug when a intermediate node indicating a disease different from a disease indicated by a target node exists on a path for the drug-disease node pair,
a simplified molecular-input line-entry system (SMILES)-based character string of the basic drug is input in the structure prediction model,
wherein the physical property includes at least one of solubility, hydration energy, melting point, boiling point, toxicity, electrical stability, excited state property, protein-ligand binding, dissociation constant, and membrane permeability,
the physical property prediction model is independently generated for each of the physical properties,
the physical property prediction model is a classification model or a regression model, and
the learning about the physical property prediction model is performed by applying substances with known physical properties and physical properties of the substances as inputs and outputs, respectively.

13. The computing apparatus of claim 12, wherein the
standard scores of proximities of the drug-disease node pairs is calculated via the following <Equation>
$$\underline{z ( s , t ) = \frac{d ( s , t ) - mean ( d ( s , T ) )}{SD ( d ( s , T ) )}} \qquad \underline{\text{<} Equation \text{>}}$$
wherein the s is a source node indicating drug, the t is a target node indicating disease, z(s, t)is the standard scores of proximities of the source node s and the target node t, d(s, t) is the shortest path between the source node s and the target node t, the T is a set of target nodes, the mean(d(s,T)) is the mean of the shortest paths for node pairs consisting of the source node s and the target node ser T, and the SD(d(s, T)) is the standard deviation of the shortest paths for node pairs consisting of the source node s and the target node set T,
the set of target nodes may be nodes randomly selected from the refined knowledge network.

14. The computing apparatus of claim 12, wherein the analogous substance obtaining unit converts each of characters constituting the SMILES-based character string for the basic drug into a vector of a reference size by replacing the character with an index corresponding to the character, extracts a feature of the vector by encoding the vector, and determines a reconstruction vector generated by decoding the feature as the analogous substance.

15. (canceled)
